(19) United States
(12) Patent Application Publication
(10) Pub. No.: US 2026/0325018 A1
(43) Pub. Date: Oct. 22, 2026

(54) DEVICE AND METHOD FOR MONITORING pH IN ABDOMINAL CAVITY AFTER DIGESTIVE TRACT SURGERY

(71) Applicant: THE SECOND AFFILIATED HOSPITAL ZHEJIANG UNIVERSITY SCHOOL OF MEDICINE, Hangzhou (CN)

(72) Inventors: Shaojun YU, Hangzhou (CN); Qun DENG, Hangzhou (CN); Limian LING, Hangzhou (CN); Hengchen LIU, Hangzhou (CN); Can LU, Hangzhou (CN)

(21) Appl. No.: 19/693,355

(22) Filed: May 30, 2026

(30) Foreign Application Priority Data
Nov. 13, 2025 (CN) 202511660247.0

(51) Int. Cl. A61B 5/145; A61B 5/00

(52) U.S. Cl. CPC A61B 5/14539; A61B 5/14503; A61B 5/742

(57) ABSTRACT

A device for monitoring pH in an abdominal cavity after digestive tract surgery, including a control unit, a cannula assembly, an electrode ring, a guide tube and an electrode guide wire. The electrode ring is slidably sleeved on of the cannula assembly. The guide tube penetrates through the electrode ring, and is fixedly connected with the electrode ring. The electrode guide wire penetrates through the guide tube, and is embedded inside the guide tube. A first end of the electrode guide wire penetrates through an outer side wall of the electrode ring away from the cannula assembly, and a second end of the electrode guide wire penetrates through the guide tube to be connected with the control unit. A method for monitoring pH in an abdominal cavity of a subject in need thereof is also provided, which is performed based on such device.

### CROSS-REFERENCE TO RELATED APPLICATIONS

This application claims the benefit of priority from Chinese Patent Application No. 202511660247.0, filed on Nov. 13, 2025. The content of the aforementioned application, including any intervening amendments thereto, is incorporated herein by reference in its entirety.

### TECHNICAL FIELD

This application relates to medical apparatuses, and more particularly to a device and method for monitoring a pH in an abdominal cavity after digestive tract surgery.

### BACKGROUND

Abdominal cavity defines a relatively large space. Intestinal leakage refers to a condition where contents of a digestive tract leak into the abdominal cavity caused by damage or rupture of an intestinal wall. If the intestinal leakage occurs or a similar problem occurs in a stomach, it may cause severe infection and inflammation, and may lead to serious complications, such as peritonitis.

In the prior art, there is no effective method for monitoring a specific abnormal area within the abdominal cavity, and thus it fails to precisely flush the abnormal location. Moreover, the monitoring and flushing operations generally cannot be performed simultaneously, which is not conducive to the postoperative recovery.

### SUMMARY

The present disclosure provides a device and method for monitoring a pH in an abdominal cavity after digestive tract surgery, which can realize real-time monitoring of abnormalities within the abdominal cavity, and location and flushing of an abnormal area within the abdominal cavity.

A device for monitoring a pH in an abdominal cavity after digestive tract surgery is provided, comprising:
a control unit;
a cannula assembly;
an electrode ring;
a guide tube, and
an electrode guide wire;
wherein the electrode ring is slidably sleeved on a periphery of the cannula assembly; the guide tube penetrates through the electrode ring, and is fixedly connected with the electrode ring; the electrode guide wire penetrates through the guide tube, and is embedded inside the guide tube; a first end of the electrode guide wire penetrates through an outer side wall of the electrode ring away from the cannula assembly, and a second end of the electrode guide wire penetrates through the guide tube to be connected with the control unit; and the control unit is configured to be arranged in vitro.

In an embodiment, a first end of the cannula assembly is sealed, and a second end of the cannula assembly is open; the cannula assembly comprises an outer cannula and an inner cannula; the outer cannula is spaced apart from the inner cannula, and is sleeved outside the inner cannula; and an end of the outer cannula is fixedly connected and sealed with an end of the inner cannula.

In an embodiment, a periphery of the outer cannula is provided with a plurality of first through holes and a plurality of second through holes; the plurality of first through holes and the plurality of second through holes are arranged spaced apart on the periphery of the outer cannula; each of the plurality of first through holes penetrates sequentially through the outer cannula and the inner cannula, and communicates with an interior of the inner cannula; and
the plurality of first through holes and the plurality of second through holes are arranged on the first end of the cannula assembly.

In an embodiment, at least two electrode rings are provided; the at least two electrode rings are sleevedly provided on the periphery of the cannula assembly, spaced apart from each other;
at least two guide tubes are provided; and the at least two guide tubes are sleevedly provided on the periphery of the cannula assembly along the at least two electrode rings, spaced apart from each other.

In an embodiment, the number of the guide tube is larger than or equal to that of the electrode ring.

In an embodiment, the guide tube, the outer cannula and the inner cannula are flexible tubes.

A method for monitoring a pH in an abdominal cavity of a subject in need thereof after digestive tract surgery, the method being performed based on the device described above, for real-time monitoring, and the method comprising:
(S1) deploying the device in the abdominal cavity to monitor the pH in the abdominal cavity in real time; inserting the cannula assembly into the abdominal cavity; and monitoring a pH value of a surrounding environment through the electrode guide wire;
(S2) generating, by the device, a first pH monitoring curve based on data monitored by the electrode guide wire;
(S3) when a change is observed in the first pH monitoring curve, issuing an alarm and continuously monitoring the abdominal cavity by the device;
(S4) determining an abnormal pH area according to the first pH monitoring curve, and estimating an amount of water required for flushing;
(S5) obtaining a first estimated abnormal point according to a peak of the first pH monitoring curve;
(S6) activating a flushing function of the device; and moving a flushing part of the device to the abnormal pH area for flushing;
(S7) after the flushing is completed, continuously monitoring the pH in the abdominal cavity to obtain a second pH monitoring curve, and obtaining a second estimated abnormal point based on the second pH monitoring curve; moving the flushing part to the abnormal pH area, and flushing an area between the first estimated abnormal point and the second estimated abnormal point; and
(S8) examining the area between the first estimated abnormal point and the second estimated abnormal point for subsequent treatment based on an actual condition.

In an embodiment, the control unit comprises an intelligent computing system and a display module; and
step (S2) comprises:
monitoring, by the electrode guide wire, pH values of monitoring points; plotting, by the intelligent computing system, the first pH monitoring curve based on the pH values of the monitoring points; and displaying, by the display module, the first pH monitoring curve.

In an embodiment, step (S6) comprises:
sliding the cannula assembly such that a portion of the cannula assembly provided with the plurality of first through holes and the plurality of second through holes is within to the abnormal pH area; introducing flushing water to a space between the outer cannula and the inner cannula, and delivering the flushing water into the abnormal pH area through the plurality of second through holes for flushing; and after the flushing is completed, withdrawing liquid from the abnormal pH area into the inner cannula through the plurality of first through holes, and discharging the liquid out through the inner cannula.

In an embodiment, step (S6) comprises:
sliding the cannula assembly such that a portion of the cannula assembly provided with the plurality of first through holes and the plurality of second through holes is within the abnormal pH area; introducing flushing water into the inner cannula, and delivering the flushing water into the abnormal pH area through the plurality of first through holes; and after the flushing is completed, withdrawing liquid from the abnormal pH area to a space between the outer cannula and the inner cannula through the plurality of second through holes, and discharging the liquid to outside.

Compared to the prior art, the present disclosure has the following beneficial effects. The present disclosure solves the problem that a pH meter is too large to be directly placed into the abdominal cavity. The present disclosure has a wide monitoring range and enables point monitoring of pH values at multiple positions within the abdominal cavity. After intestinal leakage occurs, the present disclosure facilitates rapid positioning, so as to perform targeted flushing, which is efficient and safe, and can more thoroughly clean and disinfect an area contaminated by the intestinal leakage, while avoiding a situation where a large-scale flushing of the abdominal cavity is required, which would affect the patient's recovery. The present disclosure facilitates quickly finding an intestinal leakage point for examination. Once the intestinal leakage occurs, the alarm is issued, which is conducive to monitoring the patient's health and recovery status.

### BRIEF DESCRIPTION OF THE DRAWINGS

FIG. 1 is a flow chart of a method for monitoring a pH in an abdominal cavity of a subject in need thereof after digestive tract surgery according to an embodiment of the present disclosure.

FIG. 2 is a structural diagram of a device for monitoring the pH in the abdominal cavity after digestive tract surgery according to an embodiment of the present disclosure.

FIG. 3 is a cross-sectional diagram of the device according to an embodiment of the present disclosure.

FIG. 4 is a pH monitoring curve according to an embodiment of the present disclosure.

In the figures: 1, cannula assembly; 11, outer cannula; 12, inner cannula; 13, first through hole; 14, second through hole; 2, electrode ring; 3, guide tube; and 4, electrode guide wire.

### DETAILED DESCRIPTION OF EMBODIMENTS

The present disclosure will be further described below with reference to the embodiments, and the embodiments are only for explaining the present disclosure, rather than limiting the present disclosure.

### Embodiment 1

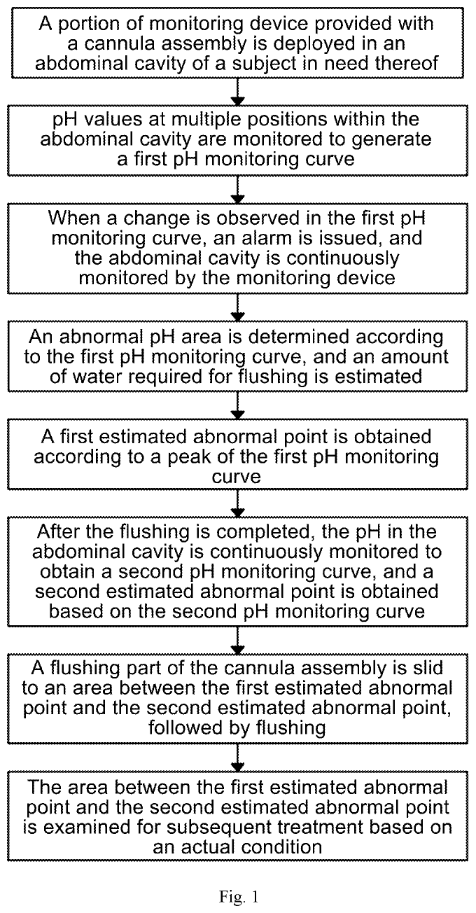

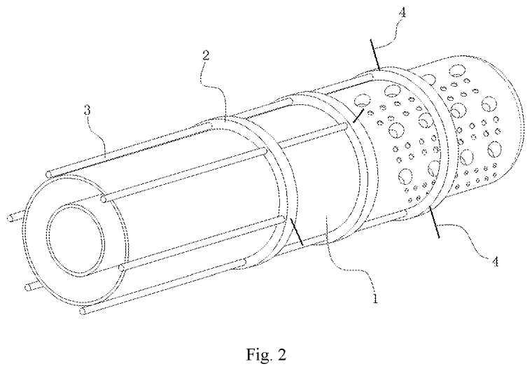

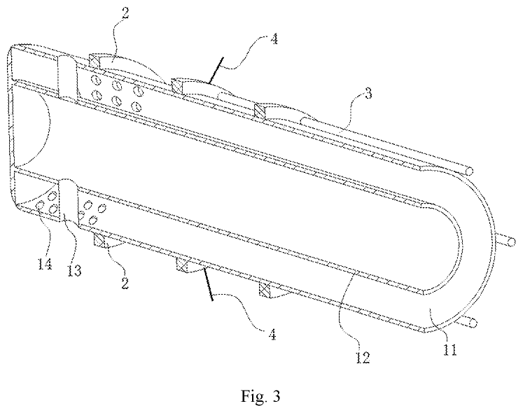

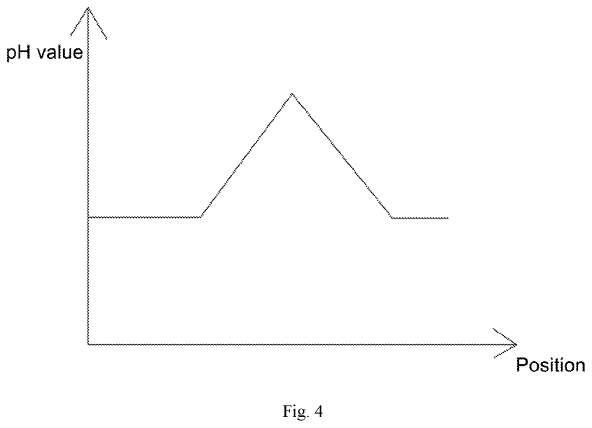

Referring to FIGS. 1-4, a method for monitoring a pH in an abdominal cavity of a subject in need thereof after digestive tract surgery is provided, and the method being performed based on a device for monitoring the pH in the abdominal cavity. The method includes the following steps.
(S1) The device is deployed in the abdominal cavity to monitor the pH in the abdominal cavity in real time.

In an embodiment, the device includes a control unit, a cannula assembly 1, an electrode ring 2, a guide tube 3 and an electrode guide wire 4. The electrode ring 2 is slidably sleeved on a periphery of the cannula assembly 1. The guide tube 3 penetrates through the electrode ring 2, and is fixedly connected with the electrode ring 2. The electrode guide wire 4 penetrates through the guide tube 3, and is embedded inside the guide tube 3. A first end of the electrode guide wire 4 penetrates through an outer side wall of the electrode ring 2 away from the cannula assembly 1, and a second end of the electrode guide wire 4 penetrates through the guide tube 3 to be connected with the control unit. The control unit is configured to be arranged in vitro.

Step (S1) includes the following steps. The cannula assembly 1 is inserted into the abdominal cavity. A pH value of a surrounding environment is monitored by the electrode guide wire 4.
(S2) A first pH monitoring curve is generated by the device based on data monitored by the electrode guide wire 4.

In an embodiment, the control unit includes an intelligent computing system and a display module. Step (S2) includes the following steps.

pH values of monitoring points are monitored by the electrode guide wire 4. The first pH monitoring curve is plotted by the intelligent computing system based on the pH values of the monitoring points. The first pH monitoring curve is displayed by the display module.
(S3) When a change is observed in the first pH monitoring curve, an alarm is issued, and the abdominal cavity is continuously monitored by the device.

In an embodiment, when fluctuations occur in the first pH monitoring curve, an alarm component set in the control unit would issue the alarm.
(S4) An abnormal pH area is determined according to the first pH monitoring curve, and an amount of water required for flushing is estimated.

In an embodiment, based on fluctuation points in the first pH monitoring curve, data of which electrode guide wire 4 is determined. An expected abnormal pH value area is determined based on a position of the electrode guide wire 4.
(S5) A first estimated abnormal point is obtained according to a peak of the first pH monitoring curve.

In an embodiment, a corresponding electrode guide wire 4 is inferred according to the peak of the first pH monitoring curve, so as to determine a corresponding abnormal area in the abdominal cavity.
(S6) A flushing function of the device is activated. A flushing part of the device is moved to the abnormal pH area for flushing.

In an embodiment, a first end of the cannula assembly 1 is sealed, and a second end of the cannula assembly 1 is open. The cannula assembly 1 includes an outer cannula 11 and an inner cannula 12. The outer cannula 11 is spaced apart from the inner cannula 12, and is sleeved outside the inner cannula 12. An end of the outer cannula 11 is fixedly connected and sealed with an end of the inner cannula 12.

A periphery of the outer cannula 11 is provided with a plurality of first through holes 13 and a plurality of second through holes 14. The plurality of first through holes 13 and the plurality of second through holes 14 are arranged spaced apart on the periphery of the outer cannula 11. Each of the plurality of first through holes 13 penetrates sequentially through the outer cannula 11 and the inner cannula 12, and communicates with an interior of the inner cannula 12.

Step (S6) includes the following steps. The cannula assembly such that a portion of the cannula assembly provided with the plurality of first through holes 13 and the plurality of second through holes 14 is slid within the abnormal pH area.

In an embodiment, step (S6) further includes the following steps. Flushing water is introduced to a space between the outer cannula 11 and the inner cannula 12, and is delivered into the abnormal pH area through the plurality of second through holes 14 for flushing. After the flushing is completed, liquid from the abnormal pH area is withdrawn into the inner cannula 12 through the plurality of first through holes 13, and is discharged out through the inner cannula 12.

In an embodiment, step (S6) further includes the following steps. Flushing water is introduced into the inner cannula 12, and is delivered into the abnormal pH area through the plurality of first through holes 13. After the flushing is completed, liquid from the abnormal pH area is withdrawn to a space between the outer cannula 11 and the inner cannula 12 through the plurality of second through holes 14, and is discharged to outside.
(S7) After the flushing is completed, the pH in the abdominal cavity is continuously monitored to obtain a second pH monitoring curve, and a second estimated abnormal point is obtained based on the second pH monitoring curve. The flushing part is moved to the abnormal pH area, and an area between the first estimated abnormal point and the second estimated abnormal point is flushed.

In an embodiment, step (S7) includes the following steps. The cannula assembly 1 is slid, and the portion of the cannula assembly provided with the plurality of first through holes 13 and the plurality of second through holes 14 is slid to the area between the first estimated abnormal point and the second estimated abnormal point, followed by flushing.
(S8) The area between the first estimated abnormal point and the second estimated abnormal point is examined for subsequent treatment based on an actual condition.

In an embodiment, at least two electrode rings 2 are provided. The at least two electrode rings 2 are sleevedly provided on the periphery of the cannula assembly 1, spaced apart from each other.

In an embodiment, at least two guide tubes 3 a provided. The at least two guide tubes 3 are sleevedly provided on the periphery of the cannula assembly 1 along the at least two electrode rings 2, spaced apart from each other.

In an embodiment, the number of the guide tube 3 is larger than or equal to that of the electrode ring 2.

In an embodiment, the plurality of first through holes 13 and the plurality of second through holes 14 are arranged on the first end of the cannula assembly 1.

In an embodiment, the guide tube 3, the outer cannula 11 and the inner cannula 12 are flexible tubes, where the flexible tubes can be bent and will not cause harm to a human body when they extend into the abdominal cavity.

A working principle of the device is as follows.

The device is inserted into the abdominal cavity, and monitors pH values at multiple locations within the abdominal cavity through different electrode guide wires for point monitoring. Medical personnel generate a pH monitoring curve based on monitored data. If an abnormality occurs in the pH monitoring curve, the medical personnel identify which electrode guide wire's monitoring data corresponds to an abnormal pH point, thereby determining a location of the abnormal pH point within the abdominal cavity. The medical personnel can move the plurality of first through holes and the plurality of second through holes to the location. Flushing water is delivered through the plurality of first through holes or the plurality of second through holes to clean the location, and then liquid from the abnormal pH area within the abdominal cavity is removed through the plurality of first through holes and the plurality of second through holes.

Described above are merely preferred embodiments of the present disclosure, which are not intended to limit the disclosure. It should be noted that any improvements, equivalent replacements or modifications made by those of ordinary skill in the art within the spirit of the present disclosure, shall fall within the scope of this application defined by the appended claims.

What is claimed is:

1. A device for monitoring a pH in an abdominal cavity after digestive tract surgery, comprising:
a control unit;
a cannula assembly;
an electrode ring;
a guide tube, and
an electrode guide wire;
wherein the electrode ring is slidably sleeved on a periphery of the cannula assembly; the guide tube penetrates through the electrode ring, and is fixedly connected with the electrode ring; the electrode guide wire penetrates through the guide tube, and is embedded inside the guide tube; a first end of the electrode guide wire penetrates through an outer side wall of the electrode ring away from the cannula assembly, and a second end of the electrode guide wire penetrates through the guide tube to be connected with the control unit; and the control unit is configured to be arranged in vitro.

2. The device of claim 1, wherein a first end of the cannula assembly is sealed, and a second end of the cannula assembly is open; and
the cannula assembly comprises an outer cannula and an inner cannula; the outer cannula is spaced apart from the inner cannula, and is sleeved outside the inner cannula; and an end of the outer cannula is fixedly connected and sealed with an end of the inner cannula.

3. The device of claim 2, wherein a periphery of the outer cannula is provided with a plurality of first through holes and a plurality of second through holes; the plurality of first through holes and the plurality of second through holes are arranged spaced apart on the periphery of the outer cannula; each of the plurality of first through holes penetrates sequentially through the outer cannula and the inner cannula, and communicates with an interior of the inner cannula; and
the plurality of first through holes and the plurality of second through holes are arranged on the first end of the cannula assembly.

4. The device of claim 1, wherein at least two electrode rings are provided; the at least two electrode rings are sleevedly provided on the periphery of the cannula assembly, spaced apart from each other;
at least two guide tubes are provided; and the at least two guide tubes are sleevedly provided on the periphery of the cannula assembly along the at least two electrode rings, spaced apart from each other.

5. The device of claim 1, wherein the number of the guide tube is larger than or equal to that of the electrode ring.

6. The device of claim 2, wherein the guide tube, the outer cannula and the inner cannula are flexible tubes.

7. A method for monitoring a pH in an abdominal cavity of a subject in need thereof after digestive tract surgery, the method being performed based on the device of claim 3, and the method comprising:
(S1) deploying the device in the abdominal cavity to monitor the pH in the abdominal cavity in real time; inserting the cannula assembly into the abdominal cavity; and monitoring a pH value of a surrounding environment through the electrode guide wire;
(S2) generating, by the device, a first pH monitoring curve based on data monitored by the electrode guide wire;
(S3) when a change is observed in the first pH monitoring curve, issuing an alarm and continuously monitoring the abdominal cavity by the device;
(S4) determining an abnormal pH area according to the first pH monitoring curve, and estimating an amount of water required for flushing;
(S5) obtaining a first estimated abnormal point according to a peak of the first pH monitoring curve;
(S6) activating a flushing function of the device; and moving a flushing part of the device to the abnormal pH area for flushing;
(S7) after the flushing is completed, continuously monitoring the pH in the abdominal cavity to obtain a second pH monitoring curve, and obtaining a second estimated abnormal point based on the second pH monitoring curve; moving the flushing part to the abnormal pH area, and flushing an area between the first estimated abnormal point and the second estimated abnormal point; and
(S8) examining the area between the first estimated abnormal point and the second estimated abnormal point for subsequent treatment based on an actual condition.

8. The method of claim 7, wherein the control unit comprises an intelligent computing system and a display module; and
step (S2) comprises:
monitoring, by the electrode guide wire, pH values of monitoring points; plotting, by the intelligent computing system, the first pH monitoring curve based on the pH values of the monitoring points; and displaying, by the display module, the first pH monitoring curve.

9. The method of claim 7, wherein step (S6) comprises:
sliding the cannula assembly such that a portion of the cannula assembly provided with the plurality of first through holes and the plurality of second through holes is within the abnormal pH area; introducing flushing water to a space between the outer cannula and the inner cannula, and delivering the flushing water into the abnormal pH area through the plurality of second through holes for flushing; and after the flushing is completed, withdrawing liquid from the abnormal pH area into the inner cannula through the plurality of first through holes, and discharging the liquid out through the inner cannula.

10. The method of claim 7, wherein step (S6) comprises:
sliding the cannula assembly such that a portion of the cannula assembly provided with the plurality of first through holes and the plurality of second through holes is within the abnormal pH area; introducing flushing water into the inner cannula, and delivering the flushing water into the abnormal pH area through the plurality of first through holes; and after the flushing is completed, withdrawing liquid from the abnormal pH area to a space between the outer cannula and the inner cannula through the plurality of second through holes, and discharging the liquid to outside.
